(19) United States
(12) Patent Application Publication
(10) Pub. No.: US 2025/0057520 A1
(43) Pub. Date: Feb. 20, 2025

(54) DEVICES, SYSTEMS, AND METHODS FOR IN VIVO TISSUE DECONSTRUCTION

(71) Applicant: ROSWELL PARK CANCER INSTITUTE CORPORATION, Buffalo, NY (US)

(72) Inventor: Todd L. DEMMY, Williamsville, NY (US)

(21) Appl. No.: 18/722,040

(22) PCT Filed: Dec. 19, 2022
(86) PCT No.: PCT/US2022/053384

§ 371 Date: Jun. 20, 2024

Related U.S. Application Data
(60) Provisional application No. 63/291,651, filed on Dec. 20, 2021.

(51) Int. Cl. A61B 17/00

(52) U.S. Cl. CPC A61B 17/00234; A61B 2017/00287; A61B 2017/00292; A61B 2017/00486; A61B 2017/00907; A61B 2217/007

(57) ABSTRACT

Various implementations include an in vivo tissue deconstruction system. The system includes an adapter, a deconstruction sack, and a circulation catheter. The adapter has a first end, a second end, and an adapter opening extending from the first end to the second end. The deconstruction sack includes a fluid-impermeable sheet, an inner volume, and a mouth providing access to the inner volume. The deconstruction sack is movable from a collapsed configuration to an expanded configuration. The mouth of the deconstruction sack is sealingly couplable to the adapter. The circulation catheter includes an irrigation conduit and a drainage conduit. The circulation catheter is disposed within the adapter opening and the inner volume of the deconstruction sack. The irrigation conduit of the circulation catheter is fluidically couplable to a supply of deconstruction fluid such that the inner volume of the deconstruction sack is in fluid communication with the supply of deconstruction fluid.

### BACKGROUND

Physicians often need to remove tissue structures/surgical specimens from patients for health and diagnostic reasons such as microscopic evaluations. Extraction of a surgical specimen such as an organ traditionally requires creation of a larger wound than the wound needed to disconnect it from surrounding tissue. In many operations, this larger wound is also traumatized by stretching forces. Such a wound can create unwanted cosmetic results and can cause pain if there is prolongation of convalescence. Further, compared to the wound needed to disconnect a surgical specimen from surrounding tissue, traditional wounds for extraction of a surgical specimen can create increased risk of complications like pneumonia because of the effects larger wounds have on respiratory mechanics.

Current microscopic evaluation systems, only require small amounts of organ tissue to be processed by a pathologist and the remaining tissue is often fixed in formalin then discarded after the samples are obtained. As such, the current standard procedure requires delivery of organs intact to the pathologist for examination to determine important characteristics relating to disease biology (e.g., tumors). However, new pathology standard procedures are becoming more popular, which provide new diagnostic capabilities. With new procedures, the current quality standard for obtaining small samples from critical areas of the organ specimen could be maintained even though the surgeon takes the sample instead of the traditional pathologist.

Thus, there exists a need for a minimally invasive system and method to remove organs from a living patient.

### SUMMARY

Various implementations include an in vivo tissue deconstruction system configured for passage through a minimally invasive opening to deconstruct a tissue structure with a supply of deconstruction fluid. The system includes an adapter, a deconstruction sack, and a circulation catheter. The adapter has a first end and a second end opposite the first end. The first end of the adapter defines an adapter opening extending from the first end of the adapter to the second end of the adapter. The deconstruction sack includes a fluid-impermeable sheet. The deconstruction sack defines an inner volume and includes a mouth providing access to the inner volume. The deconstruction sack is movable from a collapsed configuration to an expanded configuration. The deconstruction sack has a smaller cross-section in the collapsed configuration than in the expanded configuration. The mouth of the deconstruction sack is sealingly couplable to the adapter such that the adapter opening is in fluid communication with the inner volume of the deconstruction sack. The circulation catheter includes an irrigation conduit and a drainage conduit. The circulation catheter is disposed within the adapter opening with a distal tip of the circulation catheter being disposed within the inner volume of the deconstruction sack such that the irrigation conduit and the drainage conduit are in fluid communication with the inner volume of the deconstruction sack. The irrigation conduit of the circulation catheter is fluidically couplable to a supply of deconstruction fluid such that the inner volume of the deconstruction sack is in fluid communication with the supply of deconstruction fluid.

In some implementations, the deconstruction sack further includes an inner layer and an outer layer. In some implementations, the inner layer and the outer layer are each independently sealingly couplable to an outer surface of the adapter. In some implementations, the inner layer includes reinforced nylon. In some implementations, the outer layer includes neoprene. In some implementations, at least a portion of the outer layer is transparent or translucent.

In some implementations, the adapter includes an outer tapered portion that tapers from a largest diameter closest to the second end of the adapter to a smallest diameter closest to the first end. In some implementations, the deconstruction sack further includes an inner layer and an outer layer, and the outer layer is sealingly coupled to the outer tapered portion. In some implementations, the adapter defines an excess fluid channel extending from a first channel end defined by the outer surface of the tapered portion to a second channel end defined by a portion of the outer surface of the adapter between the tapered portion and the second end of the adapter.

In some implementations, the system further includes a reservoir that defines an inner volume. In some implementations, the inner volume is configured to be in fluidic communication with the inner volume of the deconstruction sack. In some implementations, the inner volume of the reservoir is configured to contain deconstruction fluid. In some implementations, the reservoir is configured to cause the circulation of deconstruction fluid from the inner volume of the reservoir into the deconstruction sack and from the deconstruction sack into the inner volume of the reservoir. In some implementations, the inner volume of the reservoir is in fluid communication with the irrigation conduit and the drainage conduit of the circulation catheter. In some implementations, the reservoir further includes a tissue collection filter disposed within the inner volume of the reservoir. In some implementations, deconstruction fluid circulating from the deconstruction sack into the inner volume of the reservoir flows through the tissue collection filter. In some implementations, the tissue collection filter retains bodily tissue flowing within the circulating deconstruction fluid. In some implementations, the tissue collection filter is removably coupled to the reservoir. in some implementations, the tissue collection filter has a mesh size from 0.02 mm to 5.0 mm.

In some implementations, the reservoir further includes an outflow filter disposed within the inner volume of the reservoir. In some implementations, deconstruction fluid circulating from the inner volume of the reservoir to the inner volume of the deconstruction sack flows through the tissue collection filter. In some implementations, the outflow filter retains bodily tissue flowing within the circulating deconstruction fluid.

In some implementations, the system further includes an outflow pump for causing the flow of deconstruction fluid through the irrigation conduit.

In some implementations, the system further includes an inflow pump for causing the flow of deconstruction fluid through the drainage conduit.

In some implementations, the system further includes an inflow pump for causing a low-pressure region within the inner volume of the reservoir relative to the inner volume of the deconstruction sack.

In some implementations, the system further includes a timer controller for activating and deactivating the one or more pumps.

In some implementations, the system further includes an agitation device for causing movement of deconstruction fluid within the deconstruction sack. In some implementations, the agitation device includes a pressure device for causing deconstruction fluid to be moved in and out of the deconstruction sack. In some implementations, the agitation device includes a balloon coupled to the circulation catheter. In some implementations, the balloon is inflatable to cause deconstruction fluid within the deconstruction sack to be moved.

Various other implementations include a method of removing tissue from within a patient. The method includes providing a deconstruction sack as described above, inserting the deconstruction sack through a minimally invasive opening in a patient while the deconstruction sack is in the collapsed configuration, moving the deconstruction sack to the expanded configuration, disposing a tissue structure within the inner volume, sealingly coupling the mouth of the deconstruction sack about an adapter as described above, and inserting a circulation catheter within the adapter opening as described above.

In some implementations, the method further includes causing deconstruction fluid to flow from the irrigation conduit into the deconstruction sack and from the deconstruction sack into the drainage conduit after inserting the circulation catheter within the adapter opening.

In some implementations, the method further includes moving the deconstruction sack to the collapsed position after inserting the circulation catheter within the adapter opening.

In some implementations, the method further includes removing the deconstruction sack from the patient through the minimally invasive opening after inserting the circulation catheter within the adapter opening.

In some implementations, the method further includes forming the minimally invasive opening in the patient before inserting the deconstruction sack.

In some implementations, the method further includes disconnecting the tissue structure inside the patient from surrounding tissue before disposing the tissue structure within the inner volume.

In some implementations, the deconstruction sack further includes an inner layer and an outer layer. In some implementations, the inner layer and the outer layer are each independently sealingly couplable to an outer surface of the adapter. In some implementations, the inner layer includes reinforced nylon. In some implementations, the outer layer includes neoprene. In some implementations, at least a portion of the outer layer is transparent or translucent.

In some implementations, the adapter includes an outer tapered portion that tapers from a largest diameter closest to the second end of the adapter to a smallest diameter closest to the first end. In some implementations, the deconstruction sack further includes an inner layer and an outer layer, and the outer layer is sealingly coupled to the outer tapered portion. In some implementations, the adapter defines an excess fluid channel extending from a first channel end defined by the outer surface of the tapered portion to a second channel end defined by a portion of the outer surface of the adapter between the tapered portion and the second end of the adapter.

In some implementations, the method further includes a reservoir that defines an inner volume. In some implementations, the inner volume is configured to be in fluidic communication with the inner volume of the deconstruction sack. In some implementations, the inner volume of the reservoir is configured to contain deconstruction fluid. In some implementations, the reservoir is configured to cause the circulation of deconstruction fluid from the inner volume of the reservoir into the deconstruction sack and from the deconstruction sack into the inner volume of the reservoir. In some implementations, the inner volume of the reservoir is in fluid communication with the irrigation conduit and the drainage conduit of the circulation catheter. In some implementations, the reservoir further includes a tissue collection filter disposed within the inner volume of the reservoir. In some implementations, deconstruction fluid circulating from the deconstruction sack into the inner volume of the reservoir flows through the tissue collection filter. In some implementations, the tissue collection filter retains bodily tissue flowing within the circulating deconstruction fluid. In some implementations, the tissue collection filter is removably coupled to the reservoir. in some implementations, the tissue collection filter has a mesh size from 0.02 mm to 5.0 mm.

In some implementations, the reservoir further includes an outflow filter disposed within the inner volume of the reservoir. In some implementations, deconstruction fluid circulating from the inner volume of the reservoir to the inner volume of the deconstruction sack flows through the tissue collection filter. In some implementations, the outflow filter retains bodily tissue flowing within the circulating deconstruction fluid.

In some implementations, the method further includes an outflow pump for causing the flow of deconstruction fluid through the irrigation conduit.

In some implementations, the method further includes an inflow pump for causing the flow of deconstruction fluid through the drainage conduit.

In some implementations, the method further includes an inflow pump for causing a low-pressure region within the inner volume of the reservoir relative to the inner volume of the deconstruction sack.

In some implementations, the method further includes a timer controller for activating and deactivating the one or more pumps.

In some implementations, the method further includes providing an agitation device for causing movement of deconstruction fluid within the deconstruction sack. In some implementations, the agitation device includes a pressure device for causing deconstruction fluid to be moved in and out of the deconstruction sack. In some implementations, the agitation device includes a balloon coupled to the circulation catheter, the balloon being inflatable to cause deconstruction fluid within the deconstruction sack to be moved.

### BRIEF DESCRIPTION OF THE DRAWINGS

Example features and implementations are disclosed in the accompanying drawings. However, the present disclosure is not limited to the precise arrangements and instrumentalities shown.

FIG. 1 shows a side view of an example in-vivo tissue deconstruction system, according to one implementation.

FIG. 2 shows a side view of the adapter of the system shown in FIG. 1.

FIG. 3A shows a side view of a deconstruction sack of the system of FIG. 1 inserted in a patient and surrounding an organ of the patient.

FIG. 3B shows a side view of the deconstruction sack of FIG. 3A coupled to an adapter.

FIG. 3C shows a side view of a circulation catheter inserted through the adapter and into the deconstruction sack.

FIG. 3D shows an enlarged side view of the circulation catheter shown in FIG. 3C.

FIG. 3E shows a side view of the deconstruction sack of FIG. 3A being removed from the patient with residual portions of the organ.

FIG. 4 is a chart showing a rate of dissolution of a liver using Potassium Chloride or Sodium Hydroxide.

FIG. 5 shows experimental results of intracorporal tissue digestion utilizing alkaline hydrolysis.

FIG. 6 shows a side view of an example in-vivo tissue deconstruction system, according to another implementation.

FIG. 7 shows a side view of an example in-vivo tissue deconstruction system, according to another implementation.

FIGS. 8A-8D show perspective views of the process of coupling a deconstruction sack to an adapter, according to some implementations.

### DETAILED DESCRIPTION

The devices, systems, and methods disclosed herein provide a means to dissolve at least a portion of tissue within a bodily cavity and remove the tissue through a minimally invasive opening in the bodily cavity without spillage. The implementations described herein provide systems and methods for using endoscopic surgery for organ removal to reduce incision size in patients. These implementations described herein also provide a way for a user to achieve better quantification of tissue sample attributes and, therefore, to better understand diseases. The deconstruction system is passable through a minimally invasive opening in a patient to deconstruct a tissue structure such as an organ with a supply of deconstruction fluid while protecting surrounding tissue. The system provides for tissue removal and tissue sample collection without creating a large invasive wound or opening in the organism.

Various implementations include an in vivo tissue deconstruction system configured for passage through a minimally invasive opening to deconstruct a tissue structure with a supply of deconstruction fluid. The system includes an adapter, a deconstruction sack, and a circulation catheter. The adapter has a first end and a second end opposite the first end. The first end of the adapter defines an adapter opening extending from the first end of the adapter to the second end of the adapter. The deconstruction sack includes a fluid-impermeable sheet. The deconstruction sack defines an inner volume and includes a mouth providing access to the inner volume. The deconstruction sack is movable from a collapsed configuration to an expanded configuration. The deconstruction sack has a smaller cross-section in the collapsed configuration than in the expanded configuration. The mouth of the deconstruction sack is sealingly couplable to the adapter such that the adapter opening is in fluid communication with the inner volume of the deconstruction sack. The circulation catheter includes an irrigation conduit and a drainage conduit. The circulation catheter is disposed within the adapter opening with a distal tip of the circulation catheter being disposed within the inner volume of the deconstruction sack such that the irrigation conduit and the drainage conduit are in fluid communication with the inner volume of the deconstruction sack. The irrigation conduit of the circulation catheter is fluidically couplable to a supply of deconstruction fluid such that the inner volume of the deconstruction sack is in fluid communication with the supply of deconstruction fluid.

Various other implementations include a method of removing tissue from within a patient. The method includes providing a deconstruction sack as described above, inserting the deconstruction sack through a minimally invasive opening in a patient while the deconstruction sack is in the collapsed configuration, moving the deconstruction sack to the expanded configuration, disposing a tissue structure within the inner volume, sealingly coupling the mouth of the deconstruction sack about an adapter as described above, and inserting a circulation catheter within the adapter opening as described above.

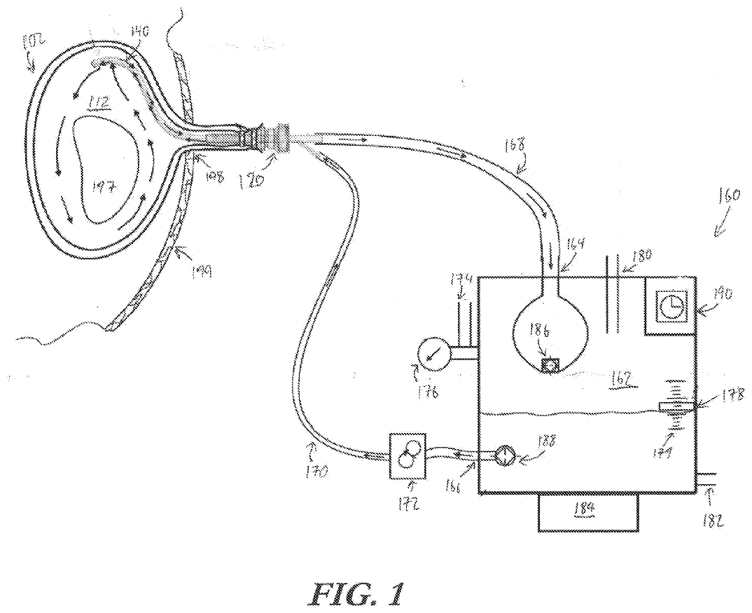

FIG. 1 shows an example in-vivo tissue deconstruction system 100. The system 100 shown in FIG. 1 includes a deconstruction sack 102, an adapter 120, a circulation catheter 140, and a reservoir 160.

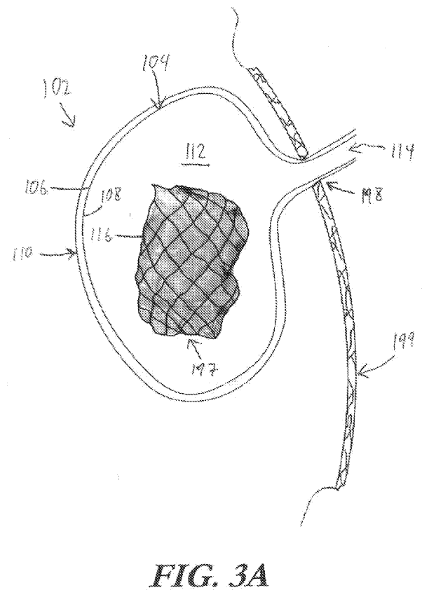

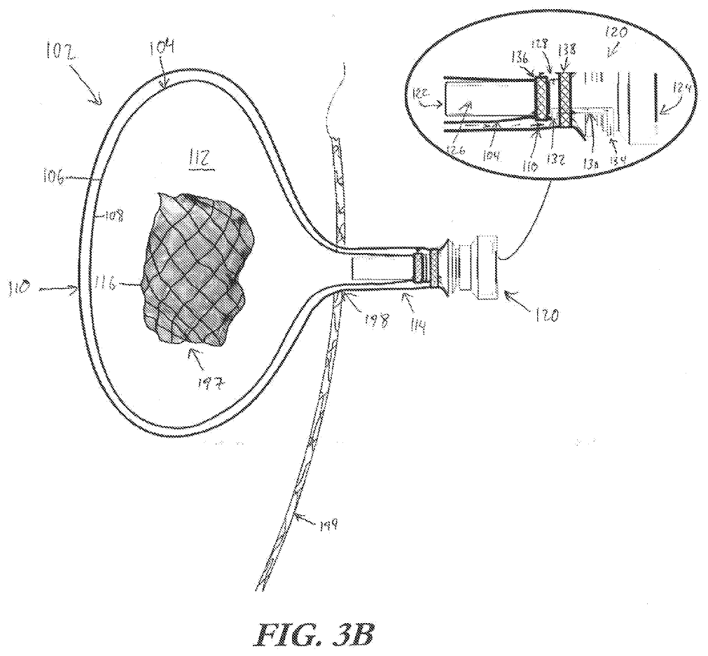

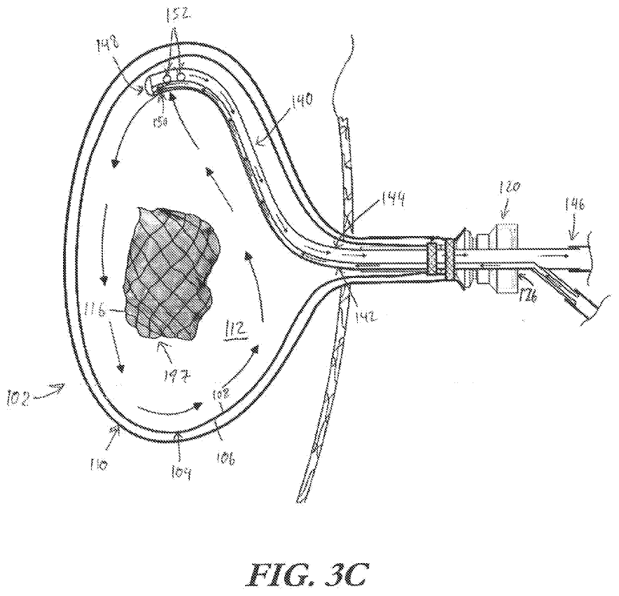

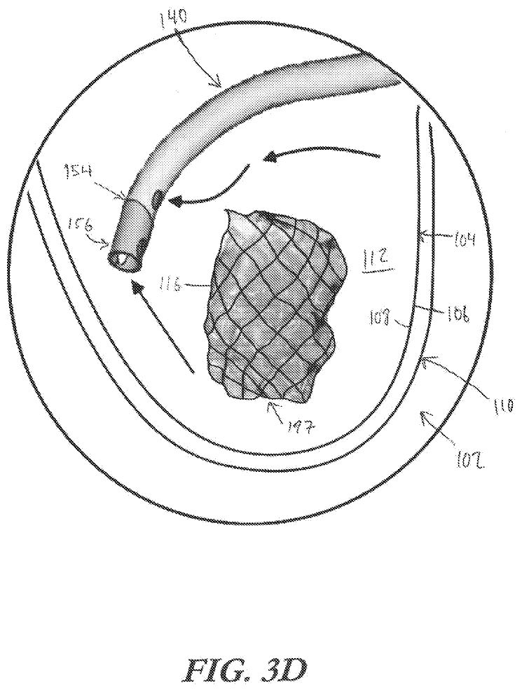

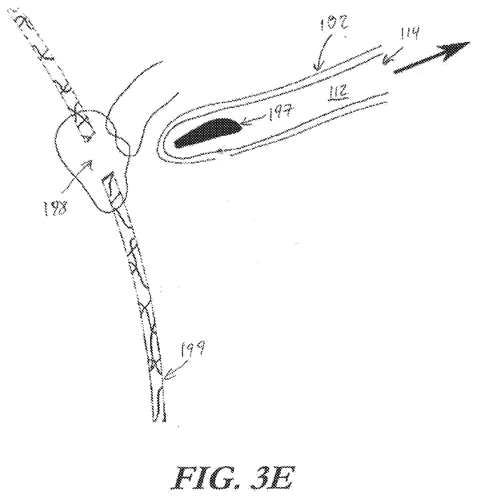

The deconstruction sack 102 is provided to surround a portion of tissue, such as an organ, and isolate the tissue from the surrounding environment, such as a cavity in a patient's body. The deconstruction sack 102 is substantially impermeable to deconstruction fluid (described further below). The deconstruction sack 102 shown in FIGS. 1 and 3A-3E includes an inner layer 104 having an outer surface 106 and inner surface 108, and an outer layer 110 abutting the outer surface 106 of the inner layer 104. The inner surface 108 of the inner layer 104 defines an inner volume 112. The inner layer 104 and the outer layer 110 form a mouth 114 that is in fluidic communication with the inner volume 112.

The deconstruction sack 102 is made of a flexible material and is movable from an expanded configuration to a collapsed configuration. When the deconstruction sack 102 is in the collapsed configuration, the deconstruction sack 102 has a minimal cross-section such that the deconstruction sack 102 can be inserted through a minimally invasive opening 198 of an organism 199, such as a person. When collapsed, the deconstruction sack 102 can be rolled into a compact cylinder to be introduced into a body cavity. Once the deconstruction sack 102 is at least partially inserted through the minimally invasive opening 198, the deconstruction sack 102 can be moved to the expanded configuration such that the inner surface 108 of the inner layer 104 of the deconstruction sack 102 defines an inner volume 114 that can surround the tissue structure 197 to be removed.

In some implementations, the inner layer 104 is opaque, and the outer layer 110 is transparent or translucent, such that fluid between the inner layer 104 and the outer layer 110 is visible through the outer layer 110 of the deconstruction sack 102. The transparency of the outer layer 110 allows a user to determine if fluid leakage has occurred through the inner layer 104 of the deconstruction sack 102. The inner layer 104 and the outer layer 110 of the deconstruction sack 102 shown in FIGS. 1 and 3A-3E are partially coupled together such that the inner layer 104 and the outer layer 110 are separate at the mouth 114 of the deconstruction sack 102. As such, the inner layer 104 and the outer layer 110 are each independently sealable against separate sealing surfaces of the adapter 120, as described below.

The deconstruction sack 102 is foldable such that the deconstruction sack 102 retains a folded form when in the collapsed configuration and retains an expanded form in the expanded configuration when the deconstruction sack 102 is at least partially filled with fluid. In the collapsed configuration, the deconstruction sack 102 shown in FIGS. 1 and 3A-3E has a length of 254 mm, a width of 230 mm and a thickness of 0.25 mm. However, in other implementations, the deconstruction sack can have a length within the range of 100 mm-300 mm] and a width within the range of 100 mm-200 mm such that the deconstruction sack is capable of enveloping and retaining a desired portion of tissue from an organism and circulating fluid thereabout. The inner layer 104 is made of nylon with a polyurethane coating on the inner surface 108 that is configured to be impermeable to fluid such as bodily fluid and deconstruction fluid. The outer layer 110 is made of transparent neoprene.

Although the inner layer 104 shown in FIGS. 1 and 3A-3E is made of nylon with a polyurethane layer coating on the inner surface 108, in other implementations, the inner layer can be any impermeable, foldable material capable of retaining bodily fluid and deconstruction fluid. In other implementations, the inner surface of the inner layer can be coated with any flexible hydrophobic material that can form a bonded layer along the inner surface of the deconstruction sack. Although the outer layer 110 shown in FIGS. 1 and 3A-3E is made of neoprene, in other implementations, the outer layer can be any impermeable, foldable material capable of retaining bodily fluid and deconstruction fluid. Although the inner layer 104 and the outer layer 110 shown in FIGS. 1 and 3A-3E are partially coupled together such that the inner layer 104 and the outer layer 110 are separate at the mouth 114 of the deconstruction sack 102, in other implementations, the inner layer and the outer layer are not coupled together.

As shown in FIGS. 3A-3D, the deconstruction sack 102 also includes a meshed organ constraint 116 that is disposed within the inner volume 104 of the deconstruction sack 102. The meshed organ constraint 116 is provided to maintain a distance between the tissue structure 197 and circulation catheter 140 to improve drainage of the deconstruction fluid and prevent expansion of dissolving tissue (caused by osmotic, vascular, and intraorgan infusion effects), thereby preventing large pieces of tissue, such as pieces of an organ, from occluding the drainage holes 152 in the circulation catheter 140.

The meshed organ constraint 116 shown in FIGS. 3A-3D is made of a polymer material and can be located within the inner volume 104 of the deconstruction sack 102 when the deconstruction sack 102 is initially inserted through the minimally invasive opening 198 of the organism 199. The tissue structure 197 can then we disposed within the meshed organ constraint 116, and the meshed organ constraint 116 can be fastened around the tissue structure 197 using surgical stapling. However, in other implementations, the meshed organ constraint is not disposed within the deconstructions sack when the deconstruction sack is initially inserted through the minimally invasive opening of the organism, and the meshed organ constraint is inserted through the minimally invasive opening separately from the deconstruction sack. In some implementations, the meshed organ constraint is not tightly coupled around the tissue structure as shown in FIGS. 3A-3D and loosely encloses the tissue structure. In some implementations, the meshed organ constraint is made of any other suitable material for being placed within a deconstruction sack within an organism and can have any size mesh openings to act as a filter to only allow deconstructed tissue of a specified size from passing through the mesh openings. In some implementations, the system does not include a meshed organ constraint.

The adapter 120 has a first end 122 and a second end 124 opposite the first end 122. The first end 122 of the adapter 120 defines an adapter opening 126 extending from the first end 122 of the adapter 120 to the second end 124 of the adapter 120. The first end 122 of the adapter 120 provides a sealed channel to secure the inner layer 104 and the outer layer 110 of the deconstruction sack 102 at a point outside a patient's body. Attaching the deconstruction sack 102 outside a patient's body is more comfortable than attaching the deconstruction sack 102 inside the patient's body.

The adapter 120 shown in FIGS. 1, 2, 3B, and 3C includes an outer tapered portion 128 that tapers axially from a largest diameter closest to the second end 124 of the adapter 120 to a smallest diameter closest to the first end 122. However, in other implementations, the first end can include other elongated shapes, such as cylindrical or cuboid shapes.

As shown in FIG. 3B, the adapter 120 further defines an excess fluid channel 130. The excess fluid channel 130 extends from a first channel end 132 defined by the outer surface of the tapered portion 128 to a second channel end 134 defined by a portion of the outer surface of the adapter 120 between the tapered portion 128 and the second end 124 of the adapter 120.

The adapter 120 includes an inner seal 136 and an outer seal 138. The inner seal 136 is disposed about an outer surface of the adapter 120 between the first end 122 of the adapter 120 and the tapered portion 128, and the outer seal 138 is disposed about an outer surface of the tapered portion 128 of the adapter 120. The inner seal 136 and the outer seal 138 are disposed such that the first channel end 132 is disposed axially between the inner seal 136 and the outer seal 138, and the second channel end 134 is disposed axially between the outer seal 138 and the second end 124 of the adapter 120. The inner seal 136 is configured to sealingly couple the inner layer 104 of the deconstruction sack 102 to the adapter 120, and the outer seal 138 is configured to sealingly couple the outer layer 110 of the deconstruction sack 102 to the tapered portion 128 of the adapter 120, providing a redundant seal.

Because the tapered portion 128 of the adapter 120 to which the outer seal 138 is coupled includes a larger diameter than the diameter of the portion of the outer surface of the adapter 120 to which the inner seal 136 is coupled, the mouth 114 of the inner layer 104 and the outer layer 110 are radially spaced apart from each other. This spacing of the inner layer 104 and the outer layer 110 of the deconstruction sack 102 allows any excess fluid present between the inner layer 104 and the outer layer 110 to freely flow toward the outer seal 138. The presence of fluid at the outer seal 138 would indicate leakage from the inner layer 104 into the space between the inner layer 104 and outer layer 110. Because the excess fluid channel 130 provides fluid communication from the space between the inner layer 104 and outer layer 110 to a location external to the deconstruction sack 102, the presence of fluid flowing through the excess fluid channel 130 and out of the second channel end 134 indicates the presence of fluid leakage between the inner layer 104 and outer layer 110 and can alert a physician to take appropriate measures to prevent further fluid leakage through the outer layer 110 into a patient.

Although the inner seal 136 and the outer seal 138 shown in FIGS. 1, 3B, and 3C are each an elastic seal, in other implementations, the inner seal and the outer seal can be a screw seal, a clamp seal, or any other seal capable of securing and fluidically sealing a foldable sack about a tubular device.

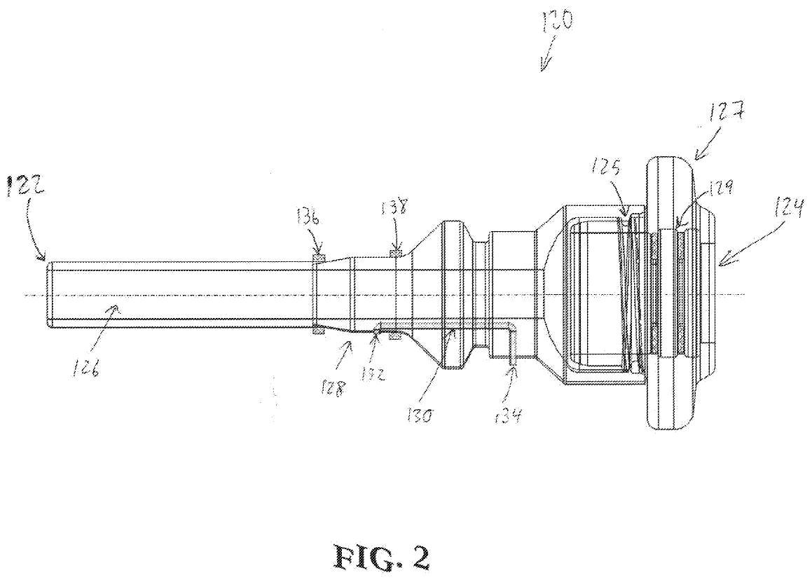

The adapter 120 shown in FIG. 2 also include a removal cap 127 that includes the second end 124 of the adapter 120 and defines a portion of the adapter opening 126. The removal cap 127 includes a threaded portion 125 that threadingly couples the removal cap 127 to the main body of the adapter 120. In use, the removal cap 127 of the adapter 120 can be threadingly decoupled from the main body of the adapter 120 to provide for a larger opening in the adapter 120 if needed, for example, when the adapter opening 126 becomes clogged or a larger medical device needs to be inserted inside of the deconstruction sack 102.

The removal cap 127 also includes a catheter seal 129 sized to form a liquid-tight seal around the circumference of a circulation catheter 140 when a circulation catheter 140 is inserted within the adapter opening 126, as discussed below. The catheter seal 129 is made of a resilient material and can have a slightly smaller diameter than the diameter of the circulation catheter 140 such that insertion of the circulation catheter 140 through the catheter seal 129 urges the catheter seal 129 radially outwardly. However, in other implementations, the catheter seal can be made of any material and have any diameter opening. In other implementations, the catheter seal can be any kind of seal or valve for preventing deconstruction fluid from flowing out of the adapter opening when the circulation catheter is inserted into the adapter opening. In some implementations, the adapter does not include a catheter seal or includes two or more catheter seals or a combination of seals.

The circulation catheter 140 includes an irrigation conduit 142 and a drainage conduit 144 at least partially adjacent the irrigation conduit 142 such that the respective longitudinal axes of the irrigation conduit 142 and the drainage conduit 144 are substantially parallel, as shown in FIG. 3C. The circulation catheter 140 is insertable through the adapter opening 126 and into the deconstruction sack 102 to circulate deconstruction fluid into and out of the deconstruction sack 102. The irrigation conduit 142 is provided to release deconstruction fluid into the deconstruction sack 102. The drainage conduit 144 is provided to remove deconstruction fluid and deconstructed tissue from the inner volume 112 of the deconstruction sack 102. The circulation catheter 140 shown in FIGS. 1, 3C, and 3D is a modified 28F chest tube. The 28F chest tube is modified such that the irrigation conduit 142 and the drainage conduit 144 are each separate conduits that extend between a proximal end 146 and a distal end 148 of the circulation catheter 140. The circulation catheter 140 extends through the adapter opening 126 such that the distal end 148 of the circulation catheter 140 is disposed inside the inner volume 112 of the deconstruction sack 102 to circulate deconstruction fluid into and out of the deconstruction sack 102.

The circulation catheter 140 shown in FIGS. 1, 3C, and 3D includes one irrigation hole 150 and two drainage holes 152 defined by sides of the circulation catheter 140. But in other implementations, the circulation catheter can include any number of holes suitable to provide irrigation fluid into the deconstruction sack and remove deconstruction fluid from the deconstruction sack. Although the circulation catheter 140 shown in FIGS. 1, 3C, and 3D is a modified 28F chest tube, in other implementations, the circulation catheter can be any other sized catheter capable of providing deconstruction fluid into, suctioning the deconstruction fluid from, the inner volume of the deconstruction sack. The circulation catheter 140 is formed from flexible plastic, but in other implementations, the circulation catheter can be formed form any other material suitable to retain and direct deconstruction fluid.

In some implementations, the circulation catheter 140 also includes a catheter adapter 154 disposed at the distal end 148 of the circulation catheter 140 for coupling a medical instrument, such as devices for disrupting or extracting small quantities of tissue using endoscopic means. This adapter will be bonded with medical grade adhesive or integrated into the extrusion of the circulation catheter to reduce the possibility of release into the sac. In the implementation shown in FIG. 3D, a microdebrider device 156 is coupled to catheter adapter 154. The microdebrider device 156 includes a high-speed rotor which facilitates tissue disruption of organ elements that are aspirated into its opening. However, in other implementations, another additional medical instrument can be coupled to the catheter adapter in situations where irrigation-based methods cannot remove a tissue structure in a desired amount of time due to the composition of the tissue or the nature of an operation. Other example attachments include multi-conduit dissolution irrigation ports, ultrasonic cavitation tissue disruptors, intraorgan irrigation catheters, high local pressure fluid disruptors, thermal coupled energy, Microwave and RFA energy delivery, vibration, and intraorgan gas generation. Because the manipulations are carried out within the deconstruction sack 102, the catheter adapter 154 provides a means for introducing additional medical instruments without requiring additional major anesthetic resources.

Furthermore, in instances where other medical instruments are necessary, the circulation catheter can be removed from the deconstruction sack and from the adapter opening and a different medical instrument can be inserted through the adapter opening to perform a task within the inner volume of the deconstruction sack. For example, an endoscope can be at least partially disposed inside the inner volume of the deconstruction sack through the adapter opening. The endoscope can include features such as an extraction device or an ablation device. For example, the endoscope can include a tissue disruptor provided to move portions of tissue or provide initial separation of portions of tissue to aid chemical deconstruction.

The reservoir 160 is provided to retain deconstruction fluid to be pumped into the deconstruction sack 102 and to collect deconstruction fluid circulated through the deconstruction sack 102. The reservoir 160 is an enclosure that that defines an inner volume 162. The reservoir 160 includes an inlet port 164 and an outlet port 166. An inlet tube 168 is coupled to the inlet port 164 and to the drainage conduit 144 of the circulation catheter 140 such that the inlet port 168 and the drainage conduit 144 are in fluid communication with each other. An outlet tube 170 is coupled to the outlet port 166 and to the irrigation conduit 142 of the circulation catheter 140 such that the outlet port 166 and the irrigation conduit 142 are in fluid communication with each other. Thus, the reservoir 160 is in fluidic communication with the inner volume 112 of the deconstruction sack 102 via the inlet tube 168 and the outlet tube 170.

The reservoir 160 includes an outflow pump 172 for circulating deconstruction fluid from the reservoir 160 to the inner volume 112 of the deconstruction sack 102. The outflow pump 172 is a medical irrigation pump, although in other implementations, the outflow pump can be any type of pump capable of circulating fluid from a fluid reservoir to an outlet. The outflow pump 172 is disposed at a location along the outlet tube 170 between the outlet port 166 of the reservoir 160 and the irrigation conduit 142 of the circulation catheter 140. When the outflow pump 172 is actuated, the outflow pump 172 causes deconstruction fluid within the reservoir 160 to flow from the inner volume 162 of the reservoir 160, through the outlet port 166, through the outlet tube 170, through the irrigation conduit 142, and into the inner volume 112 of the deconstruction sack 102.

The reservoir 160 further includes an inflow vacuum source 174 for creating a vacuum within the reservoir 160 to cause circulation of deconstruction fluid from the inner volume 104 of the deconstruction sack 102 back to the reservoir 160. The inflow vacuum source 174 is suction port in a hospital room, although in other implementations, the inflow pump can be any type of pump capable of creating a negative pressure within the inner volume of the reservoir. In some implementations, the reservoir does not include an inflow vacuum source and fluid flows back through the drainage conduit to the reservoir passively (e.g., gravity) or by the suction caused by the suction side of the outflow pump. Because the inflow vacuum source 174 creates a lower pressure within the reservoir 160 than within the inner volume 112 of the deconstruction sack 102, the inflow vacuum source 174 causes deconstruction fluid within the inner volume 112 of the deconstruction sack 102 to flow from the deconstruction sack 102, through the drainage conduit 144, through the inlet tube 168, through the outlet port 166, and back into the reservoir 160. The inflow vacuum source 174 also includes a regulator and gauge 176 for selectively adjusting the pressure within the inner volume 162 of the reservoir 160.

In some implementations, the fluid flow rate of the outflow pump is equal to the fluid flow rate of the inflow pump, such that deconstruction fluid is pumped into and out of the deconstruction sack at a uniform rate and the volume of deconstruction fluid within the deconstruction sack remains constant. However, in other implementations, the outflow pump and the inflow pump operate using different flow rates or flow patterns. For example, in some implementations, the outflow pump operates at a greater flow rate than the inflow pump such that the deconstruction sack gradually fills with fluid over time. In another example, the outflow pump can run intermittently with the inflow pump such that the deconstruction sack is intermittently filled with deconstruction fluid and emptied of deconstruction fluid.

The reservoir 160 includes a volume level float 178 that indicates the amount of fluid present within the inner volume 162 of the reservoir 160 and indicates the volume of tissue deconstructed and circulated into the reservoir 160. The volume level float 178 is a buoyant float that remains atop the deconstruction fluid within the reservoir 160. The volume level float 178 can be seen through a transparent portion of the reservoir 160, and volume markers 179 are indicated on the transparent portion of the reservoir 160 such that the change in the initial float position and the float position at the end of a procedure can be determined. This change in volume of fluid within the reservoir 160 indicates the volume of deconstructed tissue present within the volume of deconstruction fluid.

The reservoir 160 also includes an additive port 180 for adding deconstruction fluid or any other desired additives for processing of a tissue structure 197 inside the deconstruction sack 102. The reservoir 160 further includes a reservoir drain 182 for removing deconstruction fluid or any other fluids from the reservoir 160 for disposal, analysis, or any other suitable use. In some examples, the reservoir 160 also includes a heater 184 configured to warm the deconstruction fluid to a desired temperature. The warming from the heater 184 can enhance the properties of the deconstruction fluid as well as minimize chances of hypothermia of a living patient being operated upon using the system 100. As shown in FIG. 1, the heater 184 can be placed against a surface of the reservoir 160 such that the deconstruction fluid is heated through conduction.

The reservoir 160 further includes a tissue collection filter 186 for retaining solid tissue recovered from the drainage conduit 144 through the inlet port 164 and allow deconstruction fluid and dissolved tissue to pass therethrough into the inner volume 162 of the reservoir 160. The tissue collection filter 186 is a mesh filter disposed within the inner volume 162 of the reservoir 160 downstream from the inlet port 164. Any solid tissue within the returning deconstruction fluid is retained in the tissue collection filter 186 to keep undesired tissue from reaching and clogging the outlet port 166 of the reservoir 160 and to allow the tissue to be removed and sampled for diagnostic purposes.

The tissue collection filter 186 shown in FIG. 1 has a mesh size of 0.1 mm, but in other implementations, the mesh size is any size from 0.02 mm to 5 mm or any other size capable of retaining bodily tissue for diagnostic testing and allow fluid to pass therethrough. In some implementations, multiple filters of different sizes are used to capture tissue samples of different sizes. For example, when specific cell populations are sought for study, mesh sizes can range from 0.02 mm to 0.1 mm.

The reservoir 160 also includes an outflow filter 188. The outflow filter 188 is a mesh filter disposed between the irrigation conduit 142 and the inner volume 162 of the reservoir 160. The outflow filter 188 shown in FIG. 1 has a mesh size capable of preventing bodily tissue from flowing from the inner volume of the reservoir to the irrigation conduit. The mesh size of the outflow filter 188 allows deconstruction fluid to flow through the outflow filter 188 and prevents the flow of tissue structure therethrough (similar to the tissue collection filter, as described above) such that residual tissue not retained by the tissue collection filter 186 does not pass back into the irrigation conduit 142 and into the deconstruction sack 102.

The reservoir 160 shown in FIG. 1 further includes a timer controller 190. The timer controller 190 is configured to activate and deactivate the outflow pump 172 and the inflow vacuum source 174 based on predetermined times. The timer controller 190 includes an electric switch (e.g., a relay switch) to control a power source to the outflow pump 172 and the inflow vacuum source 174. However, in other examples, the timer controller can be any timer capable of indicating to a user when a desired time interval has passed or capable of activating/deactivating the inflow pump and the outflow pump after a predetermined amount of time. Although the timer controller 190 shown in FIG. 1 is hard wired to control the power supply to the outflow pump 172 and inflow vacuum source 174, in other implementations, the timer controller can send a signal to the outflow pump and the inflow pump to cause the inflow pump and outflow pumps to activate and/or deactivate. In these implementations, the timer controller can send the control signal via wired communication or wireless signals such as Bluetooth, ZigBee, or any other signal capable of relaying activation instructions to an electronic pump. In the implementation shown in FIG. 1, the outflow pump 172 and the inflow vacuum source 174 are activated and deactivated simultaneously such that the amount of fluid that flows into the deconstruction sack 102 is correspondingly removed from the deconstruction sack 102. However, in some implementations, the inflow pump is activated after outflow pump has been activated, allowing the deconstruction sack to fill with a desired amount of deconstruction fluid before the deconstruction fluid is removed from the deconstruction sack by the inflow pump.

In the implementations described above, the deconstruction fluid is a composite fluid comprising about 5% Potassium Chloride. But in other implementations, the deconstruction fluid is a fluid that includes Potassium Chloride or Sodium Hydroxide or any other fluid suitable to break down a tissue structure, such as a human organ, into a substantially fluid state. As shown in FIG. 4, in an example procedure using a small quantity of liver and 250 cc of deconstruction fluid, approximately ⅔ of the tissue can be dissolved in 1 hour using only 5% hydroxide.

As described above, the deconstruction system 100 can be used to remove a tissue structure 197 from an organism 199 such as a human patient through a minimally invasive opening 198. FIGS. 3A-3E show example steps for removal of a tissue structure 197 from a human patient using the system 100 of FIG. 1. A minimally invasive opening 198 is first formed in a body cavity wall 199 of a patient. The minimally invasive opening 198 has a width large enough to receive a folded deconstruction sack 102 that is sized retain a desired tissue structure 197 (e.g., an organ as shown in the example shown in FIGS. 3A-3E) but that is as small of an opening as possible to reduce recovery time and minimize the risk of complications. The tissue structure 197 desired to be removed is then disconnected from surrounding tissue such that the tissue structure 197 is freely movable within the bodily cavity 199. The tissue structure 197 can be disconnected from surrounding tissue using any minimally invasive surgical method including utilization of robotics. These minimally invasive surgical methods can also be used to dissect and disconnect a target organ from other anatomic attachments including vascular structures.

FIG. 3A shows the deconstruction sack 102 after it has been inserted into the patient 199 through the minimally invasive opening 198. While the deconstruction sack 102 is disposed within the body cavity of the patient 199, the tissue structure 197 to be deconstructed is inserted through the mouth 114 of the deconstruction sack 102 such that the tissue structure 197 is disposed within the inner volume 112 of the deconstruction sack 102. The tissue structure 197 can be inserted into the deconstruction sack 102 by a physician using instruments such as laparoscopic instrumentation, thoracoscopic instrumentation, or robotics. Once the tissue structure 197 is disposed within the deconstruction sack 102, the mouth 114 of the deconstruction sack 102 is inserted from the body cavity 199 back through the minimally invasive opening 198 such that the mouth 114 of the deconstruction sack 102 is disposed external to the body cavity 199 while the portion of the deconstruction sack 102 containing the tissue structure 197 remains within the body cavity 199.

The first end 122 of the adapter 120 is then inserted into the mouth 114 of the deconstruction sack 102, as shown in FIG. 3B. The inner layer 104 of the deconstruction sack 102 is coupled to the adapter 102 by the inner seal 136 to fluidically seal the inner layer 104 to the adapter 120 such that the only opening to the inner volume 112 of the deconstruction sack 102 is through the adapter opening 126. The outer layer 110 of the deconstruction sack 102 is coupled to the tapered portion 128 of the adapter 120 by the outer seal 138 to fluidically seal the outer layer 110 to the adapter 120 such that the only opening to space between the inner layer 104 and the outer layer 110 of the deconstruction sack 102 is through the excess fluid channel 130.

As shown in FIG. 3C, the circulation catheter 140 is then inserted through the adapter opening 126 and into the inner volume 112 of the deconstruction sack 102 disposed within the body cavity 199. The timer controller 190 is set to actuate the outflow pump 172 and the inflow vacuum source 174 for an amount of time that has been predetermined to dissolve a desired amount of the tissue structure 197.

The inflow vacuum source 174 and outflow pump 172 cause deconstruction fluid to flow from the inner volume 162 of the reservoir 160, through the irrigation conduit 142, out of the one or more irrigation holes 150 in the circulation catheter 140, and into the inner volume 112 of the deconstruction sack 102. The deconstruction fluid circulates through the deconstruction sack 102 to deconstruct the tissue structure 197. The relatively lower pressure of the drainage conduit 144 in the circulation catheter 140 cause the deconstruction fluid to flow back through the one or more drainage holes 152 in the circulation catheter 140, through the drainage conduit 144, and into the tissue collection filter 186. The tissue collection filter 186 separates any larger portions of the captured tissue structure 197 from the deconstruction fluid while allowing the deconstruction fluid to flow through the tissue collection filter 186 and back into the inner volume 162 of the reservoir 160. The returned deconstruction fluid can then be recirculated back into the deconstruction sack 102 as described above.

In some implementations, such as the implementation shown in FIG. 3D, the distal end 148 of the circulation catheter 140 includes a catheter adapter 154 that couples a medical instrument to the circulation catheter 140. The implementation shown in FIG. 3D includes a microdebrider device 156 coupled to the catheter adapter 154, which includes a high-speed rotor for further facilitating tissue disruption of the tissue structure 197.

Once a sufficient amount of the tissue structure 197 has been deconstructed, the deconstruction sack 102 is removed from the patient 199 through the minimally invasive opening 198, as shown in FIG. 3E. Any non-dissolved, captured tissue structure 197 can be collected from the tissue collection filter 186 and removed from the reservoir 160. The captured tissue structure 197 can be analyzed for biopsy, diagnostic purposes, or any other suitable medical purpose. In some situations, the tissue structure 197 has been reduced to a size that can be removed through the minimally invasive opening 198, and the deconstruction sack 102 containing the non-deconstructed tissue structure 198 can be removed through the minimally invasive opening 197, as shown in FIG. 3E.

The minimally invasive opening 197 can then be sutured closed as necessary. For example, abdominal wounds can be closed by tying a suture using local anesthesia if necessary. However, in some situations, such as human chest wounds, the minimally invasive opening does not require closure in general operations and can heal secondarily.

FIG. 6 shows an example in-vivo tissue deconstruction system 200, according to some implementations. The system 200 shown in FIG. 6 is similar to the system 100 shown in FIG. 1 except the system 200 shown in FIG. 6 does not include a closed loop fluid system that cycles through a reservoir 160 and includes an agitation bladder 282. Because many of the features of the system 200 shown in FIG. 6 are similar to those shown in FIG. 1, similar reference numbers as those used for the system 100 shown in FIG. 1 are used to refer to similar features of the system 200 shown in FIG. 6.

The reservoir 260 of the system 200 shown in FIG. 6 defines an inner volume 262, an outlet port 266, and an additive port 280. The reservoir 260 of the system 200 shown in FIG. 6 is intended to only contain new deconstruction fluid since the deconstruction fluid is not intended to be cycled back through the reservoir 260.

A first tube 270 has a first end 272 and a second end 274. The first end 272 of the first tube 270 is fluidically coupled to the outlet port 266 of the reservoir 260 and is in fluid communication with the inner volume 262 of the reservoir 260. The first tube 270 includes a first valve 276 that is movable from an open position in which deconstruction fluid can flow through the first valve 276 and a closed position in which deconstruction fluid is prevented from flowing through the first valve 276. The second end 274 of the first tube 270 is fluidically coupled to the irrigation conduit 142 of the circulation catheter 140 such that dissolution fluid can flow from the reservoir 260, through the irrigation conduit 142, through the adapter 120, and into the deconstruction sack 102, similar to the system 100 shown in FIG. 1.

A second tube 270′ has a first end 272′ and second end 274′. The first end 272′ of the second tube 270′ is fluidically coupled to the proximal end 146 of the drainage conduit 144. The second tube 270′ includes a second valve 276′ that is movable from an open position in which deconstruction fluid can flow through the second valve 276′ and a closed position in which deconstruction fluid is prevented from flowing through the second valve 276′. The second tube 270′ also includes a branch 278′, such as a Y-fitting or a T-fitting. The first end 272′ of the second tube 270′ is closer to the branch 278′ than it is to the second valve 276′.

The second end 274′ of the second tube 270′ is fluidically coupled to the agitation bladder 282. The agitation bladder 282 is disposed within a pressure chamber 284, which includes a pressure/vacuum device 286 for adding or removing pressure from the pressure chamber 284. The pressure/vacuum device 286 shown in FIG. 6 includes a compressor that creates a variable lower pressure system, but in some implementations, the pressure/vacuum device includes a bellows, balloon style pump, or any other device capable of adding or removing pressure from the pressure from the pressure chamber.

The pressure chamber 284 is configured such that, when pressure is added or removed from the pressure chamber 284, the agitation bag 282 is compressed or expanded, respectively, by the pressure. The compression and expansion of the agitation bag 282 causes the agitation bag 282 to push and pull the deconstruction fluid through the second tube 270′ and through the circulation catheter 140. This causes agitation and circulation of the deconstruction fluid within the deconstruction sack to cause faster and more even deconstruction of the tissue structure 197.

The pressure chamber 284 and the agitation sack 282 shown in FIG. 6 include a transparent portion such that a user can see deconstruction fluid within the agitation sack 282. This allows the user to determine when the fluid needs to be replaced with fresh deconstruction fluid. It also allows the user to view pieces of deconstructed tissue to be captured. However, in some implementations, one or both of the pressure chamber or agitation sack include translucent portions and/or opaque portions.

The pressure chamber 284 further includes a pressure gauge 288 to determine the pressure within the pressure chamber 284. The pressure chamber 284 can also include a Hall effect sensor to determine when the agitation sack 282 is full and to trigger another compression cycle.

A third tube 270″ has a first end 272″ and second end 274″. The first end 272″ of the third tube 270″ is fluidically coupled to the branch 278′ of the second tube 270′. The third tube 270″ includes a second valve 276″ that is movable from an open position in which deconstruction fluid can flow through the third valve 276″ and a closed position in which deconstruction fluid is prevented from flowing through the third valve 276″. The third valve 276″ allows the user to drain the deconstruction fluid and deconstructed tissue structure 197 from the system to make space for new deconstruction fluid to enter the system from the reservoir 260. The second end 274″ of the third tube 270″ is fluidically coupled to a drainage chamber 260′ for containing the used deconstruction fluid and the deconstructed tissue structure 197.

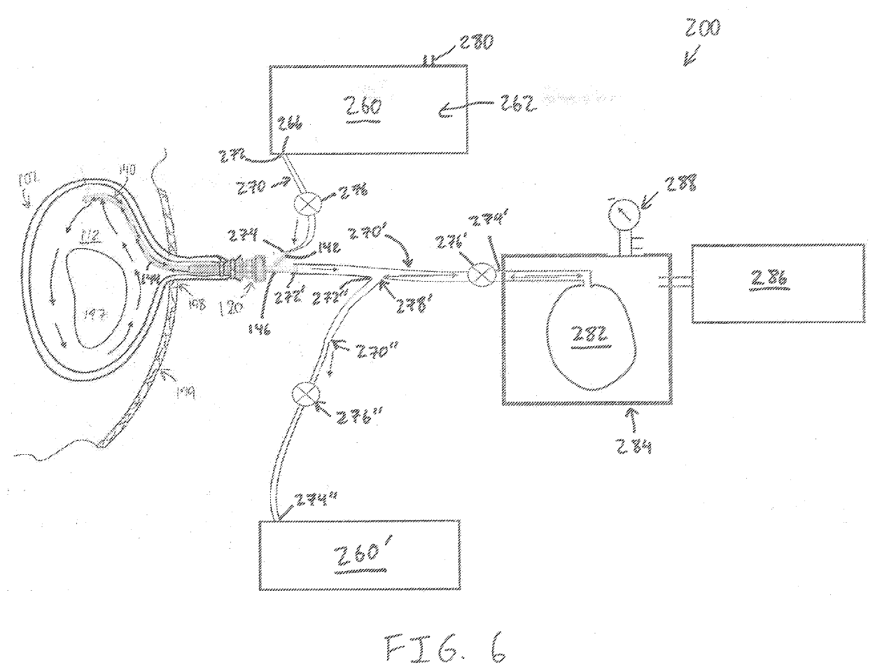

FIG. 7 shows an example in-vivo tissue deconstruction system 300, according to some implementations. The system 300 shown in FIG. 7 is similar to the system 100 shown in FIG. 1 except the circulation catheter 340 shown in FIG. 7 includes a cycling balloon 358. Because many of the features of the system 300 shown in FIG. 7 are similar to those shown in FIG. 1, similar reference numbers as those used for the system 100 shown in FIG. 1 are used to refer to similar features of the system 300 shown in FIG. 7.

The system 300 shown in FIG. 7 includes a pressure/vacuum device 386 defining a port 388. The pressure/vacuum device 386 shown in FIG. 7 includes a compressor that creates a variable lower pressure system, but in some implementations, the pressure/vacuum device includes a bellows, balloon style pump, or any other device capable of creating a variable lower pressure system.

A tube 370 has a first end 372 and a second end 374. The first end 372 of the tube 370 is fluidically coupled to the pressure/vacuum device 386, and the second end 374 of the tube 370 is fluidically coupled to the cycling balloon 358 of the circulation catheter 340 such that the pressure/vacuum device 386 is in fluid communication with the cycling balloon 358.

The cycling balloon 358 shown in FIG. 7 is 60-100 cc volume. The pressure/vacuum device 386 can move a medically safe inert low viscosity driving gas like helium through the tube 370 and into the cycling balloon 358.

When the circulation catheter 340 is disposed within the deconstruction sack 102, the pressure/vacuum device 386 can be activated to cycle between pressure and vacuum to inflate and deflate the cycling balloon 358 within the deconstruction sack 102. The movement of the cycling balloon 358 produces a fluid wave to gently break up digesting tissue structure 197 through movement of the deconstruction fluid within the deconstruction sack 102 and movement of the tissue structure 197 itself.

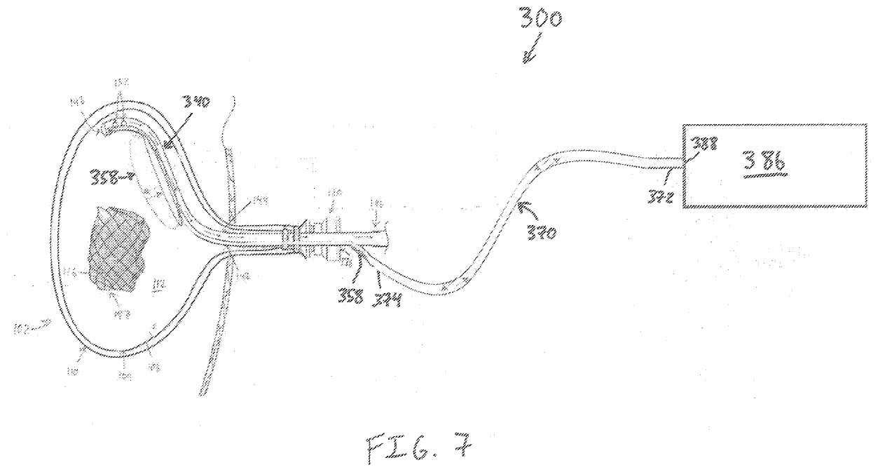

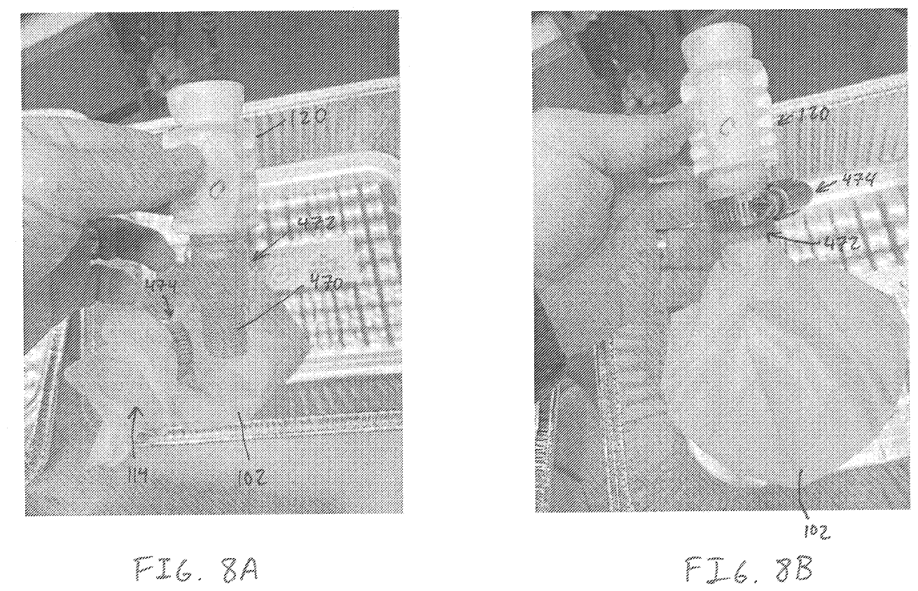

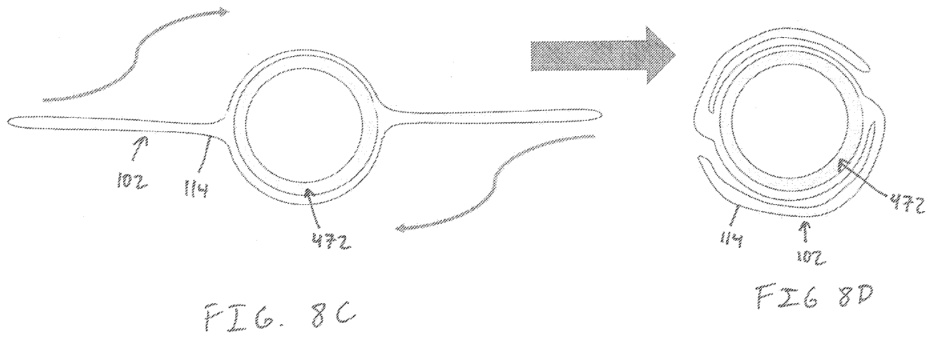

FIGS. 8A and 8B show a means for coupling the adapter 120 to the deconstruction sack 102 to prevent leaking. A length of tubing 470 is coupled to the adapter 120, and a fitting 472 is applied over the tubing 470. The fitting 472 shown in FIGS. 8A and 8B has a 0.75 inch inner diameter to fit over the 0.75 inch outer diameter tube 470 and is made of copper. However, in some implementations, the fitting is made of PVC, stainless steel, or any other rigid material. The fitting 472 is then wrapped in silicone seal adhering tape to cover the fitting 472.

Although the tubing 470 shown in FIGS. 8A and 8B is a wire reinforced tubing, the tubing 470 still lacks sufficient rigidity for sufficient compression forces to be applied to fully seal the deconstruction sack 102 onto the tubing 470. The rigidity of the fitting 472 provides a support for these compressive forces.

The mouth 114 of the deconstruction sack 102 can then be placed around the fitting 472. As shown in FIG. 8C, the mouth 114 of the deconstructions sack 102 can be flattened around the fitting 472 such that flattened portions of the mouth 114 extend from opposing sides of the fitting 472. FIG. 8D shows that the extending portions of the mouth 114 of the deconstructions sack 102 can then be wrapped in the same circumferential direction around the fitting 472 to prevent wrinkles between the deconstructions sack 102 and the fitting 472 to form a watertight seal. Another layer of silicone seal adhering tape can be applied, and a worm gear type hose clamp 474 is placed over the mouth 114 of the deconstruction sack 102 and tightened to create a watertight seal.

The system 200 shown in FIG. 6 was tested by deconstructing a lung of a pig cadaver. A port sized incision 198 was first formed in the chest wall of the pig, and the lung was placed inside a deconstructions sack 102 inside of the cadaver. 600 ml of lye was exchanged within the deconstruction sack eight times over 24 hours without agitation. After the 24 hours, the lung was examined and found to be partially digested and fragmented.

A number of implementations have been described. The description in the present disclosure has been presented for purposes of illustration but is not intended to be exhaustive or limited to the implementations disclosed. It will be understood that various modifications and variations will be apparent to those of ordinary skill in the art and may be made without departing from the spirit and scope of the claims. Accordingly, other implementations are within the scope of the following claims. The implementations described were chosen in order to best explain the principles of the vehicle trim component and a method of formation, and to enable others of ordinary skill in the art to understand the vehicle trim component for various implementations with various modifications as are suited to the particular use contemplated.

The terminology used herein is for the purpose of describing particular implementations only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, operations, elements, and/or components, but do not preclude the presence or addition of one or more other features, operations, elements, components, and/or groups thereof.

### Examples

Example methods were carried out using example systems at least partially including the aspects described above. For example, to determine whether thoracoscopic incision size and specimen extraction trauma can be reduced, intracorporal tissue digestion was studied utilizing several means including alkaline hydrolysis.

Methods: Cadaveric porcine lungs were weighed, cannulated, placed in perfusable doubled impervious 10″×8″ laparoscopic tissue extraction sacs that were submerged in water baths (370° C.). Digestion solutions were introduced by cannulating the pulmonary vessels and by bathing the tissue. Agents tested were controls (NaCl), lye (5% NaOH), enzymes (one-hour dwell of 2% trypsin, 1 mL/gm tissue or DNase, 1 mg/150 g tissue) followed by alkaline hydrolysis, and H2O2 (terminal disruption). Lungs were subjected to 6, 12, 18, 24, or 36 hours of bathing in 1000 mL of 5% NaOH. The digestion media were replaced every three hours as indicated by bath pH analyses. Digestion was quantified by comparing the tissue remaining after digestion to its initial mass. Biopsies of the parenchyma at 3-hour time points were stained with hematoxylin and eosin and assessed for structural and cytologic integrity. Containment safety was tested by allowing 1000 mL of 5% NaOH to dwell in an extraction sac for 48 hours.

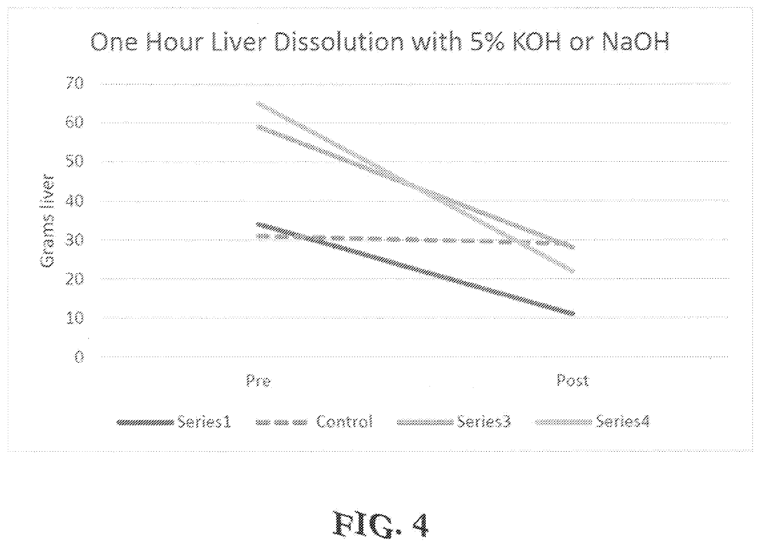

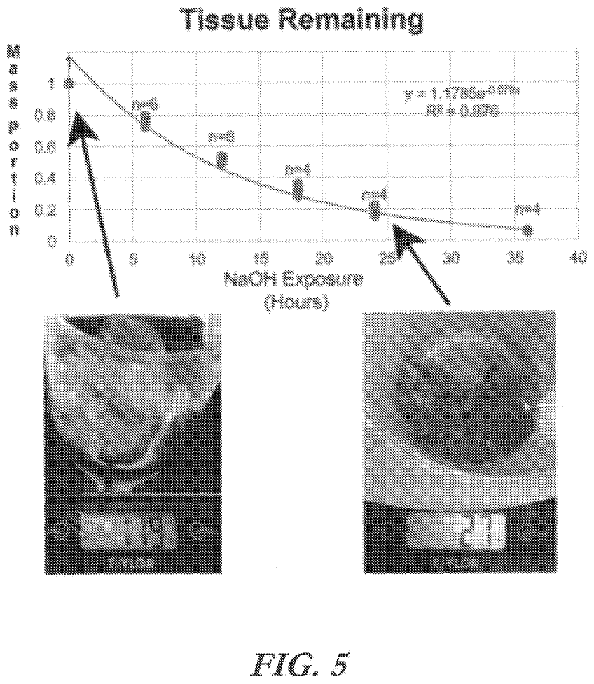

Results: A significantly smaller proportion of tissue remained following 6-hour exposure to alkaline hydrolysis with NaOH [60.9%±9.1%] compared to 6 hour exposure to NaCl [128.6%±9.6%](p<0.001). There was a significant time-dependent correlation with degeneration of parenchymal infrastructure and organization with NaOH exposure (see FIG. 5, R=0.98, p=0.02). At 36 hours, the remaining tissue was reduced to a roughly 2 cm size with a toothpaste consistency, making it amenable to removal through a surgical port (see FIG. 5). There were no containment failures.

Conclusions: These studies suggest that intracorporal tissue liquification to allow atraumatic specimen extraction through small ports is feasible during a typical inpatient hospitalization time interval.

1. An in vivo tissue deconstruction system configured for passage through a minimally invasive opening to deconstruct a tissue structure with a supply of deconstruction fluid, the system comprising:
an adapter having a first end and a second end opposite the first end, the first end of the adapter defining an adapter opening extending from the first end of the adapter to the second end of the adapter;
a deconstruction sack including a fluid-impermeable sheet, wherein the deconstruction sack defines an inner volume and includes a mouth providing access to the inner volume, the deconstruction sack being movable from a collapsed configuration to an expanded configuration, wherein the deconstruction sack has a smaller cross-section in the collapsed configuration than in the expanded configuration, wherein the mouth of the deconstruction sack is sealingly couplable to the adapter such that the adapter opening is in fluid communication with the inner volume of the deconstruction sack; and
a circulation catheter including an irrigation conduit and a drainage conduit, the circulation catheter being disposed within the adapter opening with a distal tip of the circulation catheter being disposed within the inner volume of the deconstruction sack such that the irrigation conduit and the drainage conduit are in fluid communication with the inner volume of the deconstruction sack,
wherein the irrigation conduit of the circulation catheter is fluidically couplable to a supply of deconstruction fluid such that the inner volume of the deconstruction sack is in fluid communication with the supply of deconstruction fluid.

2. The system of claim 1, wherein the deconstruction sack further comprises an inner layer and an outer layer, wherein the inner layer and the outer layer are each independently sealingly couplable to an outer surface of the adapter.

3. The system of claim 2, wherein the inner layer comprises reinforced nylon.

4. The system of claim 2, wherein the outer layer comprises neoprene.

5. The system of claim 2, wherein at least a portion of the outer layer is transparent or translucent.

6. The system of claim 1, wherein the adapter includes an outer tapered portion that tapers from a largest diameter closest to the second end of the adapter to a smallest diameter closest to the first end.

7. The system of claim 6, wherein the deconstruction sack further comprises an inner layer and an outer layer, and the outer layer is sealingly coupled to the outer tapered portion.

8. The system of claim 6, wherein the adapter defines an excess fluid channel extending from a first channel end defined by the outer surface of the tapered portion to a second channel end defined by a portion of the outer surface of the adapter between the tapered portion and the second end of the adapter.

9. The system of claim 1, further comprising a reservoir that defines an inner volume, wherein the inner volume is configured to be in fluidic communication with the inner volume of the deconstruction sack, wherein the inner volume of the reservoir is configured to contain deconstruction fluid, the reservoir being configured to cause the circulation of deconstruction fluid from the inner volume of the reservoir into the deconstruction sack and from the deconstruction sack into the inner volume of the reservoir.

10. The system of claim 9, wherein the inner volume of the reservoir is in fluid communication with the irrigation conduit and the drainage conduit of the circulation catheter.

11. The system of claim 9, wherein the reservoir further includes a tissue collection filter disposed within the inner volume of the reservoir, wherein deconstruction fluid circulating from the deconstruction sack into the inner volume of the reservoir flows through the tissue collection filter, wherein the tissue collection filter is removably coupled to the reservoir.

12. (canceled)

13. (canceled)

14. The system of claim 11, wherein the tissue collection filter has a mesh size from 0.02 mm to 5.0 mm.

15. The system of claim 9, wherein the reservoir further includes an outflow filter disposed within the inner volume of the reservoir, wherein deconstruction fluid circulating from the inner volume of the reservoir to the inner volume of the deconstruction sack flows through the tissue collection filter.

16. (canceled)

17. The system of claim 1, further comprising an outflow pump for causing the flow of deconstruction fluid through the irrigation conduit, and an inflow pump for causing the flow of deconstruction fluid through the drainage conduit.

18. (canceled)

19. The system of claim 9, further comprising an inflow pump for causing a low-pressure region within the inner volume of the reservoir relative to the inner volume of the deconstruction sack.

20. (canceled)

21. The system of claim 1, further comprising an agitation device for causing movement of deconstruction fluid within the deconstruction sack, wherein the agitation device includes a pressure device for causing deconstruction fluid to be moved in and out of the deconstruction sack, and wherein the agitation device includes a balloon coupled to the circulation catheter, the balloon being inflatable to cause deconstruction fluid within the deconstruction sack to be moved.

22. (canceled)

23. (canceled)

24. A method of removing tissue from within a patient, the method comprising:
providing a deconstruction sack including a fluid-impermeable sheet, wherein the deconstruction sack defines an inner volume and includes a mouth providing access to the inner volume, the deconstruction sack being movable from a collapsed configuration to an expanded configuration, wherein the deconstruction sack has a smaller cross-section in the collapsed configuration than in the expanded configuration;
inserting the deconstruction sack through a minimally invasive opening in a patient while the deconstruction sack is in the collapsed configuration;
moving the deconstruction sack to the expanded configuration;
disposing a tissue structure within the inner volume;
sealingly coupling the mouth of the deconstruction sack about an adapter, the adapter having a first end and a second end opposite the first end, the first end of the adapter defining an adapter opening extending from the first end of the adapter to the second end of the adapter; and
inserting a circulation catheter within the adapter opening such that a distal tip of the circulation catheter is disposed within the inner volume of the deconstruction sack, the circulation catheter including an irrigation conduit and a drainage conduit, wherein the irrigation conduit and the drainage conduit are in fluid communication with the inner volume of the deconstruction sack.

25. The method of claim 24, further comprising causing deconstruction fluid to flow from the irrigation conduit into the deconstruction sack and from the deconstruction sack into the drainage conduit after inserting the circulation catheter within the adapter opening.

26. The method of claim 24, further comprising moving the deconstruction sack to the collapsed position after inserting the circulation catheter within the adapter opening.

27. The method of claim 24, further comprising removing the deconstruction sack from the patient through the minimally invasive opening after inserting the circulation catheter within the adapter opening.

28. (canceled)

29. The method of claim 24, further comprising disconnecting the tissue structure inside the patient from surrounding tissue before disposing the tissue structure within the inner volume.

30. The method of claim 24, wherein the deconstruction sack further comprises an inner layer and an outer layer, wherein the inner layer and the outer layer are each independently sealingly couplable to an outer surface of the adapter.

31. The method of claim 30, wherein the inner layer comprises reinforced nylon, and wherein the outer layer comprises neoprene, and wherein at least a portion of the outer layer is transparent or translucent.

32. (canceled)

33. (canceled)

34. The method of claim 24, wherein the adapter includes an outer tapered portion that tapers from a largest diameter closest to the second end of the adapter to a smallest diameter closest to the first end, wherein the outer layer of the deconstruction sack is sealingly coupled to the outer taper portion.

35. (canceled)

36. The method of claim 34, wherein the adapter defines an excess fluid channel extending from a first channel end defined by the outer surface of the tapered portion to a second channel end defined by a portion of the outer surface of the adapter between the tapered portion and the second end of the adapter.

37. The method of claim 24, further comprising a reservoir that defines an inner volume, wherein the inner volume is configured to be in fluidic communication with the inner volume of the deconstruction sack, wherein the inner volume of the reservoir is configured to contain deconstruction fluid, the reservoir being configured to cause the circulation of deconstruction fluid from the inner volume of the reservoir into the deconstruction sack and from the deconstruction sack into the inner volume of the reservoir, wherein the inner volume of the reservoir is in fluid communication with the irrigation conduit and the drainage conduit of the circulation catheter.

38. (canceled)

39. The method of claim 37, wherein the reservoir further includes a tissue collection filter disposed within the inner volume of the reservoir, wherein deconstruction fluid circulating from the deconstruction sack into the inner volume of the reservoir flows through the tissue collection filter, wherein the tissue collection filter is removably coupled to the reservoir, and wherein the tissue collection filter has a mesh size from 0.02 mm to 5.0 mm.

40. (canceled)

41. (canceled)

42. (canceled)

43. The method of claim 27, wherein the reservoir further includes an outflow filter disposed within the inner volume of the reservoir, wherein deconstruction fluid circulating from the inner volume of the reservoir to the inner volume of the deconstruction sack flows through the tissue collection filter, and wherein the outflow filter retains bodily tissue flowing within the circulating deconstruction fluid.

44. (canceled)

45. The method of claim 24, further comprising an outflow pump for causing the flow of deconstruction fluid through the irrigation conduit and an inflow pump for causing the flow of deconstruction fluid through the drainage conduit.

46. (canceled)

47. The method of claim 37, further comprising an inflow pump for causing a low-pressure region within the inner volume of the reservoir relative to the inner volume of the deconstruction sack.

48. (canceled)

49. The method of claim 24, further comprising providing an agitation device for causing movement of deconstruction fluid within the deconstruction sack, wherein the agitation device includes a pressure device for causing deconstruction fluid to be moved in and out of the deconstruction sack, and wherein the agitation device includes a balloon coupled to the circulation catheter, the balloon being inflatable to cause deconstruction fluid within the deconstruction sack to be moved.

50. (canceled)

51. (canceled)
